(19) United States
(12) Patent Application Publication
(10) Pub. No.: US 2020/0060657 A1
(43) Pub. Date: Feb. 27, 2020

(54) SYSTEM AND METHOD FOR ASSESSING OBSTETRIC WELLBEING

(71) Applicant: GENERAL ELECTRIC COMPANY, Schenectady, NY (US)

(72) Inventors: Chandan Kumar Aladahalli, Bangalore (IN); Krishna Seetharam Shriram, Bangalore (IN); Rakesh Mullick, Bangalore (IN); Bipul Das, Bangalore (IN)

(21) Appl. No.: 16/109,736

(22) Filed: Aug. 22, 2018

(51) Int. Cl. A61B 8/08; G06T 7/00; G06K 9/62; G06T 7/62; G06N 3/08; G06F 16/51; G16H 50/20; G16H 30/20

(52) U.S. Cl. CPC A61B 8/5223; G06T 7/0014; G06K 9/6284; G06T 7/62; G06K 9/6256; A61B 8/0866; G06T 2207/20084; G06F 16/51; G16H 50/20; G16H 30/20; G06T 2207/10132; G06T 2207/20081; G06N 3/08

(57) ABSTRACT

A system includes a memory unit comprising a classifier network and a detector network. The classifier network is configured to perform a classification of a scan image among maternal images. The detector network is configured to determine a placenta condition in the scan image. The system further includes a data acquisition unit communicatively coupled to an ultrasound scanner and configured to receive maternal images from a maternal scanning procedure. The system also includes an image processing unit communicatively coupled to the memory unit and the data acquisition unit and configured to select a sagittal image from the maternal images using the classifier network. The image processing unit is further configured to determine a placenta condition based on the selected sagittal image using the detector network. The image processing unit is also configured to provide a recommendation to a medical professional based on the placenta condition.

### BACKGROUND

Embodiments of the present specification relates generally to the field of obstetrics, and more specifically to a method and a system for assessing obstetric conditions from maternal images acquired from a scanning procedure performed on an obstetrics subject.

Doppler ultrasound technique is a non-invasive monitoring approach to extract information about moving structures inside the body. It can be used for diagnosis of many cardiovascular conditions as well as in fetal health monitoring. Conventionally, the ultrasound technique is employed to acquire maternal data and detect a plurality of fetal parameters that may be used to determine the fetal health condition. The plurality of fetal parameters and/or fetal health condition may be displayed on a display device for the benefit of a medical professional.

Lack of standardization in acquiring maternal data makes image recognition difficult and introduces consistency issues in diagnosis. Suboptimal detection of fetal abnormalities is reported in population based studies, especially when abnormalities in complex anatomical organs, such as the fetal heart, are considered. In volume sonography, availability of 3D volumes of ultrasound image data has the potential to provide additional insights into fetal conditions. However, processing of 3D ultrasound image data also introduces several limitations. The acquisition of 3D volume, processing and display of the acquired 3D ultrasound volumes require a substantial learning curve. Volume sonography in obstetrics poses additional problems due to variable position of the fetus within the uterus. Medical professionals need enhanced technical skills to retrieve diagnostic 2D planes out of a 3D volume. The lack of standardization in the acquisition and display of 3D volumes is an impediment to training.

### BRIEF DESCRIPTION

In accordance with one aspect of the present technique a system is disclosed. The system includes a memory unit comprising a classifier network and a detector network. The classifier network is configured to perform a classification of a scan image among maternal images. The detector network is configured to determine a placenta condition in the scan image. The system further includes a data acquisition unit communicatively coupled to an ultrasound scanner and configured to receive maternal images from a maternal scanning procedure. The system also includes an image processing unit communicatively coupled to the memory unit and the data acquisition unit and configured to select a sagittal image from the maternal images using the classifier network. The image processing unit is further configured to determine a placenta condition based on the selected sagittal image using the detector network. The image processing unit is also configured to provide a recommendation to a medical professional based on the placenta condition.

In accordance with one aspect of the present technique a method is disclosed. The method includes receiving maternal images from a maternal scanning procedure. The method further includes obtaining a classifier network and a detector network from a memory unit, wherein the classifier network is configured to perform a classification of a scan image among the maternal images. The detector network is configured to determine a placenta condition in the scan image. The method also includes selecting a sagittal image from the maternal images using the classifier network. The method further includes determining a placenta condition based on the selected sagittal image using the detector network. The method also includes providing a recommendation to a medical professional based on the placenta condition.

In accordance with one aspect of the present technique a non-transitory computer readable medium having instructions to enable at least one processor unit is disclosed. The instructions enable the at least one processor unit to receive maternal images from a maternal scanning procedure. The instructions further enable the at least one processor unit to obtain a classifier network and a detector network from a memory unit, wherein the classifier network is configured to perform a binary classification of a scan image among the maternal images. The detector network is configured to determine a placenta condition in the scan image. The instructions also enable the at least one processor unit to select a sagittal image from the maternal images using the classifier network. The instructions further enable the at least one processor unit to determine a placenta condition based on the selected sagittal image using the detector network. The instructions also enable the at least one processor unit to provide a recommendation to a medical professional based on the placenta condition.

### DRAWINGS

These and other features and aspects of embodiments of the present invention will become better understood when the following detailed description is read with reference to the accompanying drawings in which like characters represent like parts throughout the drawings, wherein:

FIG. 1 is a block diagram of a system for assessing wellbeing of an obstetric subject from maternal images in accordance with one aspect of the present specification;

FIG. 2 is a flow chart of a method for assessing wellbeing of an obstetric subject from maternal images in accordance with one aspect of the present specification;

FIG. 3 is a block diagram of structure of a deep learning network used for designing one of a classification network, a detector network and a metric network in accordance with aspects of the present specification;

FIGS. 4A-4C illustrate maternal images representative of placenta conditions detected by the system of FIG. 1 in accordance with another aspect of the present specification;

FIGS. 5A-5B illustrate maternal images representative of classes of maternal images classified by the system of FIG. 1 in accordance with another aspect of the present specification;

FIGS. 6A-6C illustrate maternal images representative of fetal conditions identified by the system of FIG. 1 in accordance with another aspect of the present specification; and

FIG. 7 is a graph illustrating performance of deep learning network used for realizing classifier network and detector network in accordance with another aspect of the present specification.

### DETAILED DESCRIPTION

Embodiments of systems and methods for providing obstetric care, and more particularly to systems and methods for assessing wellbeing of an obstetric subject from maternal images are described in detail hereinafter.

The term ‘obstetric condition’ used herein refers to one of a fetal condition, a placental condition and a condition of a uterine cervix in an obstetric subject. The term ‘cephalic condition’ used herein refers to normal longitudinal position of a fetus with top of its head presented first during delivery. Any fetal condition other than the cephalic condition is generally termed herein as ‘abnormal fetal condition’. The term ‘breach condition’ refers to an abnormal fetal condition wherein at least one of buttock and feet of the fetus is presented first during the delivery. The term ‘transverse condition’ refers to an abnormal fetal position wherein the fetus is presented sideways during the child birth. The term ‘placenta’ refers to tissues that provide nourishment to and takes waste away from the fetus. The placenta is attached to inner wall of the uterus above the opening of the uterus and is connected to the fetus through the umbilical cord. The term ‘placenta condition’ refers to condition of placenta before or during the child birth. The term ‘placental abruption’ refers to a condition where placenta has begun to separate from the uterus wall. The term ‘placenta previa’ refers to a condition where the placenta lies very low in the uterus covering the opening of the uterus partially or completely. The terms ‘cervical weakness’ and ‘cervical incompetence’ are used herein equivalently and interchangeably to refer to a condition of uterine cervix failing to retain pregnancy in absence of labor causing miscarriage or preterm birth. The term ‘maternal image’ is used herein to refer to a two-dimensional (2D) scanning image obtained from an imaging modality such as an ultrasound scanner during a maternal scanning procedure. The term ‘maternal scanning’ refers to scanning of abdominal region of an obstetric subject (i.e., an expectant mother in her later stages of pregnancy). The term ‘sagittal image’ refers to a maternal image captured by transmitting ultrasound beam along a sagittal plane. The term ‘transverse image’ refers to a maternal image captured by transmitting ultrasound beam along a transverse plane. The term ‘learning network’ used herein refers generally to a deep learning network having plurality of stages of neural network based convolution layers. The term ‘classifier network’ refers to a deep learning network configured to perform a binary or a multiple label classification of an input maternal image. The term ‘detector network’ refers to a deep learning network configured to perform detection of at least one of a fetal condition, a placental condition and a cervical weakness condition in an obstetric subject based on a sagittal image. The term ‘metric network’ refers to a deep learning network configured to provide a quantification to a cervical weakness condition, a placenta condition or both based on the sagittal image. A deep learning network, such as a classifier network, a detector network and a metric network, includes a plurality of structural parameters and a plurality of network parameters which may be stored in a memory unit. The term ‘database’ used herein refers to a data structure to organize medical records and medical images. Further, the database may also be used to organize the deep learning network such as, but not limited to, a classifier network, a detector network and a metric network. Typically, the database is stored in the memory unit and operated by a management system software. The medical images may include maternal images labelled to identify a sagittal image, a fetal condition, and a placenta condition. The medical images may also be associated with one or more dimensional values (or metrics) such as, but not limited to, a uterine cervix length and a distance between the uterine cervix opening and the placenta.

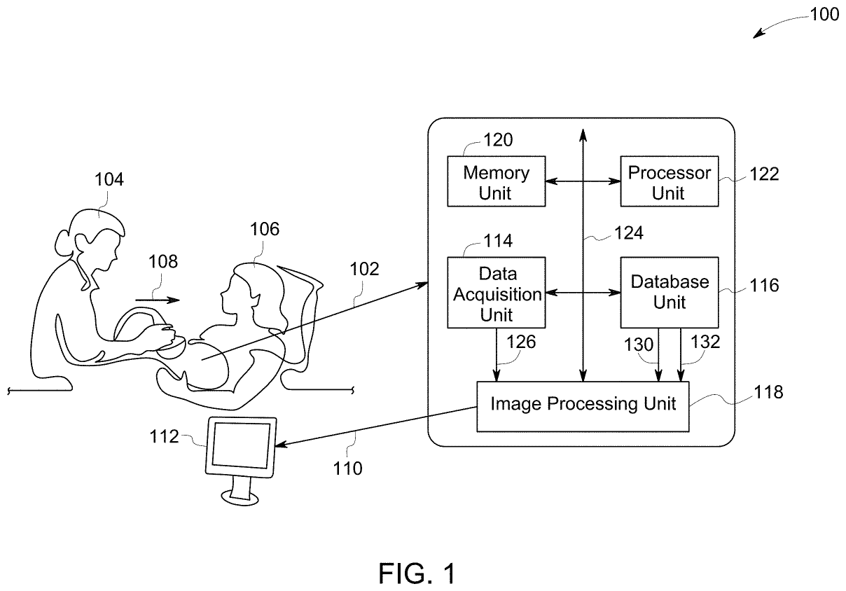

FIG. 1 is a block diagram of a system 100 for assessing wellbeing of an obstetric subject from maternal images in accordance with one aspect of the present specification. The system 100 is configured to receive a maternal image 102 from a maternal scanning procedure conducted by a medical professional 104 on an obstetric subject 106. In the illustrated embodiment, an ultrasound scanning device 108 is used to perform the maternal scanning procedure. The medical professional may include, but not limited to, a doctor, a paramedic, and a radiologist. The scanning procedure may be performed on the obstetric subject 106 either during pregnancy or during labor before delivery. The system 100 is further configured to process the received maternal image 102 and generate a recommendation 110 that may be provided to the medical professional 104. Specifically, the system 100 is configured to classify the received maternal image 102 either as a sagittal image or as a non-sagittal image. Further the system 100 is configured to process the sagittal image to detect an obstetric condition. In one embodiment, the obstetric condition is a placenta condition. In another embodiment, the obstetric condition is a fetal condition. In yet another embodiment, the obstetric condition is a cervical weakness condition. Further, the system 100 is also configured to generate a dimensional value representative of a parameter corresponding to the obstetric condition. In one example, the dimensional value corresponding to the placenta condition is representative of a distance between the placenta and opening of the uterine cervix. In another example, the dimensional value corresponding to the cervical weakness condition is a length of the uterine cervix. In the illustrated embodiment, the system 100 is communicatively coupled to a display device 112 and configured to display the one or more of the detected obstetric conditions along with corresponding dimensional values on the display device 112. The system 100 is also configured to generate a recommendation based on one or more obstetric conditions and corresponding dimensional values. In one embodiment, the system 100 is configured to operate in real time to present the recommendation to the medical professional 104.

In one embodiment, the system 100 includes a data acquisition unit 114, a database unit 116, an image processing unit 118, a memory unit 120 and a processor unit 122 communicatively coupled with each other via a communications bus 124. The image processing unit 118 is configured to receive maternal images 126 from the data acquisition unit 114 and a learning network 130 from the database unit 116 and generate the recommendation 110.

The data acquisition unit 114 is communicatively coupled to the ultrasound scanning device 108 and configured to receive the maternal images 102 acquired by the ultrasound scanning device 108 during the maternal scanning procedure conducted on the obstetric subject 106. The data acquisition unit 114 is also configured to receive parameter settings used to perform the scanning, details of the obstetric subject 106 and other scanning related data either manually from the medical professional 104 or automatically from the ultrasound scanning device 108.

The database unit 116 is communicatively coupled to the data acquisition unit 114 and configured to store maternal images received from the maternal scanning procedure. Further, the database unit 116 is also configured to store corresponding scanning parameters used to acquire the maternal images. The database unit 116 is also configured to store annotated maternal images and the annotations may include, but not limited to, class of maternal images, placenta condition, fetal condition in the content of the images. The database unit 116 may include one or more of a SQL database, an Oracle database and may be structured as a relational database, a hierarchical database, or an object-oriented database. In one embodiment, the database unit 116 is further configured to store at least one of a structural parameter and a network parameter of a classifier network designed to classify the maternal images. Further, in such an embodiment, the database unit 116 is also configured to store structural parameters and network parameters of a detector network configured to detect a placenta condition or a fetal condition. The database unit 116 is also configured to store structural parameters and network parameters of a metric network configured to determine a metric corresponding to one of the placenta condition and the fetal condition.

The image processing unit 118 is communicatively coupled to the data acquisition unit 114 and configured to process real-time maternal images 126 to generate at least one of a placenta condition or a fetal condition. The data acquisition unit 114 is also communicatively coupled to the database unit 116 and configured to receive one or more scanning parameters and annotated maternal images stored in the database unit 116. The image processing unit 118 is configured to receive one or more of the classifier network, a detector network and a metric network from the database unit 116 and/or the memory unit 120. The image processing unit 118 is configured to process the maternal images 102 obtained from the data acquisition unit 114, annotated images 132 from the database unit 116 and configured to generate the recommendation 110. The image processing unit 118 is communicatively coupled to an output device such as a display device 112 and configured to present the recommendation 110 for the benefit of the medical professional.

In one embodiment, the image processing unit 118 is configured to receive maternal images either in real-time from the data acquisition unit 114 or stored maternal images from the database unit 116. The image processing unit 118 is further configured to identify each maternal image as either a sagittal image or as a non-sagittal image using the classifier network. Further, the classified maternal image may be stored in the database unit 116 as an annotated maternal image. Further, the image processing unit 118 is configured to process a sagittal image using the detector network to identify a fetal condition. In one embodiment, the detector network is configured to detect a fetal condition of a fetus found in the sagittal image. The fetal condition includes, but not limited to, a cephalic condition (a normal condition), a breach condition or a traverse condition. In another embodiment, the detector network is configured to detect a placenta condition around a fetus found in the sagittal image. The placenta condition includes, but not limited to, a clear condition (normal condition), a low-lying condition and a previa condition. The sagittal images processed by the detector network may be stored in the database unit 116 as an annotated sagittal image. The annotations for the sagittal image may include, but not limited to, a fetal condition and/or a placenta condition around the fetus. In yet another embodiment, the image processing unit 118 is configured to process a sagittal image using the metric network to generate a dimensional parameter corresponding to one of the fetal condition and the placenta condition. The dimensional parameter corresponding to the placenta condition may be a distance value between the placenta and opening of the cervix. In another embodiment, the dimensional parameter corresponding to the cervical competency may be a length value corresponding to the cervix length.

In some embodiments, the image processing unit 118 is configured to generate the first convolutional neural network using labelled maternal images. Further, the image processing unit 118 may also be configured to generate the second convolution neural network and/or the third convolutional neural network using labelled sagittal images. Generally, the image processing unit 118 is configured to operate in an off-line mode to generate the first convolution neural network, the second convolutional neural network and the third convolution neural network. In one embodiment, labelled maternal/sagittal images are processed by the image processing unit 118 to train the learning networks. Further, trained learning networks are stored in the memory unit for future use. During on-line and/or rea-time processing, a suitable learning network, designed apriori, is retrieved from the memory and maternal images are processed by the retrieved learning network to determine a pose condition, a fetal condition and/or a placenta condition.

Specifically, in one embodiment, the image processing unit 118 is configured to receive a plurality of labelled sagittal images from the database. Each of the plurality of labelled sagittal images is annotated with a value of a dimensional parameter. Further, the image processing unit 118 is configured to train the third convolution neural learning network based on a first subset of the plurality of labelled sagittal images. The image processing unit 118 is also configured to validate the third convolution neural learning network based on a second subset of the plurality of labelled sagittal images. The image processing unit 118 is also configured to store the validated third convolution neural learning network as the metric network in the database.

In another embodiment, the image processing unit 118 is configured to design the detector network using a plurality of labelled maternal images. Specifically, the image processing unit 118 is configured to receive a plurality of labelled maternal images from the database. Each of the plurality of labelled maternal images is classified as one of a sagittal image and a non-sagittal image. The image processing unit 118 is further configured to train a first convolution neural learning network based on a first subset of the plurality of labelled maternal images and validate the first convolution neural learning network based on a second subset of the plurality of labelled maternal images. The image processing unit 118 is also configured to store the validated first convolution neural learning network as the classifier network in the database unit 116.

In yet another embodiment, the image processing unit 118 is configured to design the metric network using a plurality of annotated sagittal images. Specifically, the image processing unit 118 is configured to receive a plurality of labelled sagittal images from the database. Each of the plurality of labelled sagittal images is annotated with a fetal condition. Further, the image processing unit 118 is configured to train a second convolution neural learning network based on a first subset of the plurality of labelled sagittal images and validate the second convolution neural learning network based on a second subset of the plurality of labelled sagittal images. The image processing unit 118 is also configured to store the validated second convolution neural learning network as the detector network in the database unit 116.

In one embodiment, the image processing unit 118 is configured to receive a plurality of labelled sagittal images from the database. Each of the plurality of labelled sagittal images is annotated with a value of a dimensional parameter. Further, the image processing unit 118 is configured to train a third convolution neural learning network based on a first subset of the plurality of labelled sagittal images. The image processing unit 118 is also configured to validate the third convolution neural learning network based on a second subset of the plurality of labelled sagittal images. The image processing unit 118 is also configured to store the validated third convolution neural learning network as the metric network in the database unit 116.

The processor unit 122 is communicatively coupled to one or more of the data acquisition unit 114, the database unit 116, the image processing unit 118 and the memory unit 120 via the communications bus 124 and configured to initiate and/or control their operation. Although the processor unit 122 is shown as a separate unit, in some embodiments, the processor unit 122 may also be a part of one or more of the data acquisition unit 114, the database unit 116 and the image processing unit 118. The processor unit 122 may include one or more processors either co-located within a single circuit or distributed in multiple circuits networked to share data and communication in a seamless manner. The processor unit 122 includes at least one arithmetic logic unit, a microprocessor, a microcontroller, a general-purpose controller, a graphical processing unit (GPU) or a processor array to perform the desired functionalities or run a computer program configured to perform intended function.

The memory unit 120 is communicatively coupled to the processor unit 122 and configured to store programs, operating systems and related data required by the image processing unit 118. Although the memory unit 120 is shown as separate unit, the memory unit 120 may be part of the image processing unit 118, the data acquisition unit 114 or the database unit 116. In one embodiment, the memory unit 120 may be a dynamic random-access memory (DRAM) device, a static random-access memory (SRAM) device, flash memory or other memory devices. In another embodiment, the memory unit may include a non-volatile memory or similar permanent storage device, media such as a hard disk drive, a floppy disk drive, a compact disc read only memory (CD-ROM) device, a digital versatile disc read only memory (DVD-ROM) device, a digital versatile disc random access memory (DVD-RAM) device, a digital versatile disc rewritable (DVD-RW) device, a flash memory device, or other non-volatile storage devices. The memory unit 120 may also be a non-transitory computer readable medium encoded with a program to instruct the one or more processors to perform classification of maternal images, detect a fetal condition from a sagittal image having an image of fetus and/or detect a placenta condition from the sagittal image around the fetus.

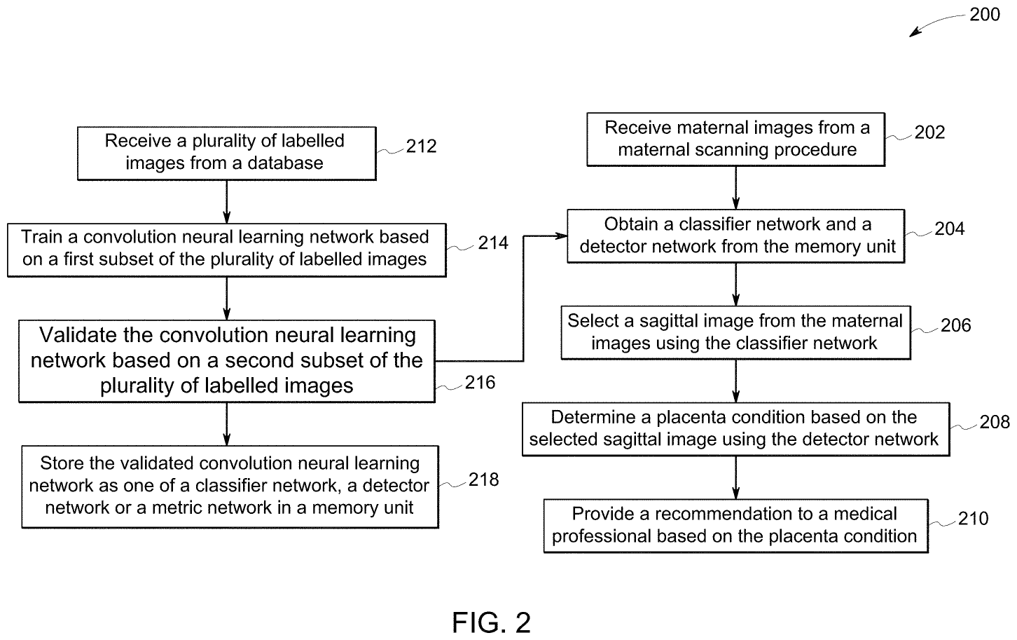

FIG. 2 is a flow chart of a method 200 for assessing wellbeing of an obstetric subject from maternal images in accordance with one aspect of the present specification. The method 200 includes receiving maternal images from a maternal scanning procedure at step 202. The maternal images include both sagittal images and non-sagittal images. The term sagittal image includes ultrasound scan images acquired in the mid-sagittal plane. The sagittal image also refers to images acquired in near vicinity of the mid-sagittal plane.

The method further includes obtaining a classifier network and a detector network from a memory unit at step 204. The classifier network is configured to perform a binary classification of a scan image among the maternal images. At step 206, the method also includes selecting a sagittal image from the maternal images using the classifier network. The selecting the sagittal image includes identifying a maternal image corresponding to a mid-sagittal plane. In another embodiment, the selecting sagittal image also includes identifying a maternal image corresponding to a plane substantially parallel to a mid-sagittal plane. The classifier network includes a first deep learning network such as, but not limited to, a convolution neural network.

The detector network is configured to determine a placenta condition in the scan image at step 208. The detector network includes a second deep learning network such as, but not limited to a convolution neural network. In one embodiment, the step 208 further includes detecting a fetal position corresponding to a breach condition, a cervical competence condition, or both by using the detector network based on the sagittal image. At step 210, the method also includes providing a recommendation to a medical professional based on the placenta condition.

In an alternate embodiment, the step 204 also includes receiving a metric network from the memory unit. The metric network is a deep learning network configured to determine a dimensional value corresponding to an obstetric condition. Specifically, when the detector network is configured to determine a placenta condition, the metric network is configured to determine a dimensional value corresponding to the placenta condition. When the detector network is configured to determine a cervical condition, the metric network is configured to determine a dimensional value corresponding to the cervical competence condition. Specifically, the step 208 in such an embodiment, includes processing the sagittal image by the metric network to determine a length value corresponding to a dimensional parameter of the obstetric condition.

It may be noted that the maternal scanning procedure performed at step 202 requires a well-trained operator capable of acquiring more number of sagittal images. In one embodiment of the present specification, the classifier network may also be employed for assisting operators having varied skills in the acquisition of sagittal images. In such an embodiment, acquiring the maternal images include performing the binary classification of the acquired maternal images as a sagittal image or as a non-sagittal image using the classifier network. Further, performing the binary classification includes determining a metric value representative of an angle formed by a plane represented by each of the maternal images with the mid-sagittal plane. The method of acquiring the maternal images is assisted by the metric value received by a medical professional performing the acquisition of maternal images.

In one embodiment, obtaining the classifier network, the detector network and optionally the metric network includes retrieving corresponding convolution neural learning network from the memory unit. The convolution neural learning network retrieved at step 204 is stored in the memory unit by performing steps 212-218 in an off-line mode. Specifically, at step 212, a plurality of labelled images is received from a database. A convolution neural learning network is trained at step 214 based on a first subset of the plurality of labelled images. In an embodiment of training the convolution neural learning network as a classifier network, the first subset includes maternal images classified as sagittal and non-sagittal images. In an embodiment of training the convolution neural learning network as a detector network, the first subset includes sagittal images labelled with an obstetric condition. In an embodiment of training the convolution neural learning network as a metric network, the first subset includes sagittal images labelled with a dimensional value corresponding to an obstetric condition.

In step 216, the trained convolution neural learning network is validated based on a second subset of the plurality of labelled images. The second subset is similar to the first subset in terms of labelling but includes maternal/sagittal images different from the images of the first subset. Further, the validated convolution neural learning network is stored in the memory unit at step 218. In one embodiment, storing the classifier network at step 218 includes training (and validating) the first convolution neural learning network based on labelled maternal images. In another embodiment, storing the detector network at step 218 includes training (and validating) the second convolution neural learning network based on labelled sagittal images. In another embodiment, storing the metric network at step 218 includes training (and validating) the third convolution neural learning network based on labelled sagittal images.

In one embodiment, at step 218, the detector network configured to detect a placenta condition is stored in the memory unit. In another embodiment, a second detector network configured to detect a fetal condition is also stored in the memory unit. Further, a third detector network configured to detect a cervical condition may also be stored in the memory unit at step 218. Similarly, a first metric network configured to determine a dimensional value corresponding to the placenta condition is stored in the memory unit. Further, a second metric network configured to determine a dimensional value corresponding to the cervical condition is also stored in the memory unit at step 218.

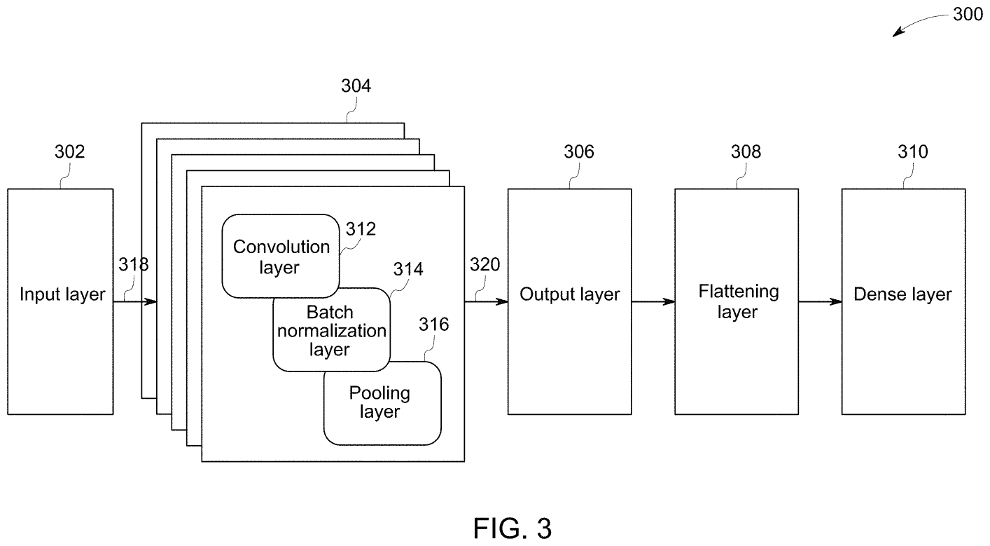

FIG. 3 is a block diagram of structure of a deep learning network 300 used for designing one or more of a classification network, a detector network and a metric network in accordance with aspects of the present specification. Specifically, the deep learning network 300 includes an input layer 302, a plurality of convolution stages 304, an output layer 306, a flattening layer 308, and a dense layer 310. Each of the plurality of convolution stages 304 includes a convolution layer 312, a batch normalization layer 314 and a pooling layer 316. In one embodiment, the deep learning network 300 having five convolution stages and trained apriori, is used to detect the placenta condition. The batch normalization layer 314 is configured to prevent overfitting and provide regularization of the classifier network. The pooling layer 316 is configured to limit the computational complexity of a subsequent stage within limits. In one embodiment, dropping of nodes is used in each convolution layer 312 as a regularization technique. In one embodiment, dropping of nodes in the convolution stages 304 is employed with a constant dropout probability value. In other embodiment, the dropping of nodes in each of the plurality of convolution stages 304 could be different. Specifically, a drop out probability value for each subsequent convolution stage may be incremented with a pre-specified value within a pre-determined range. The input layer 302 is configured to receive a two-dimensional maternal image to generate a plurality of input feature maps 318. Each of the plurality of convolution stages 304 generates a plurality of feature maps to be received by a subsequent convolution stage. The input layer 302 is a neural network configured to generate the input feature maps 318 required for the first convolution stage based on one or more input maternal (or sagittal) images. The output layer 306 is a convolution layer configured to generate output feature maps (not shown). The flattening layer 308 is configured to generate a feature vector based on the output feature maps. The dense layer 310 is a classifier network configured to provide one or more classifier outputs. A constant dropout probability may be used in the dense layer 310. The detector network is configured to generate a discrete value representative of a placenta condition at the output of the dense layer 310.

In one embodiment, learning network similar to the FIG. 3 is also used as a detector network configured to determine fetal condition. In another embodiment, a similar learning network may also be used for determining cervical condition. The classifier network and a metric network discussed in previous paragraphs may also be realized using the structure of FIG. 3. In these embodiments, a different number of convolution stages, a different number of feature maps, and other values of network parameters may be used while training the learning network.

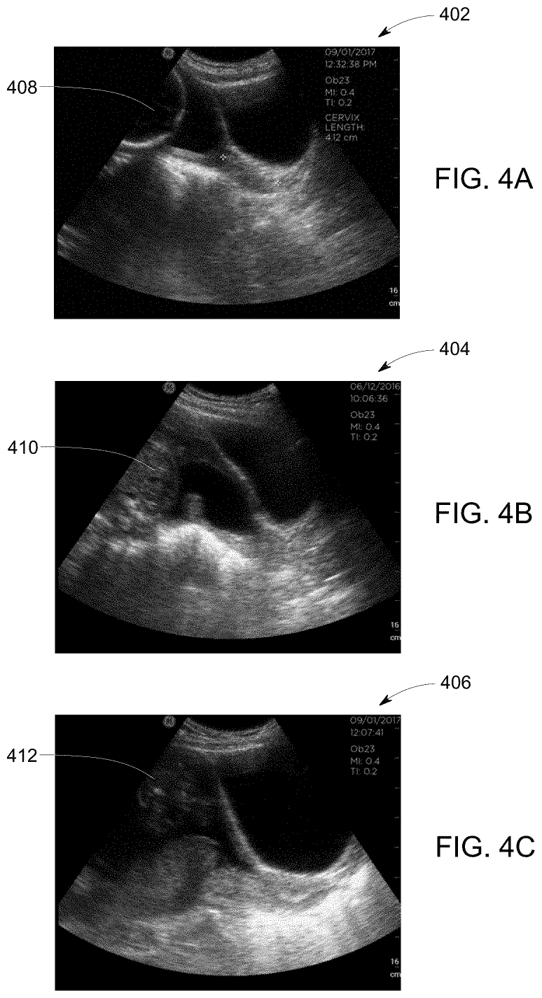

FIGS. 4A-4C illustrate maternal images representative of placenta conditions detected by the system 100 of FIG. 1 in accordance with another aspect of the present specification. The detector network used for determining the placenta condition is similar to the structure illustrated in FIG. 3. Suitable network parameters of the detector network are selected and/or determined during training phase. In this example, the detector network is a learning network designed/generated apriori using a plurality of annotated sagittal images. FIG. 4A is maternal image 402 identified by the system 100 as representative of clear placenta condition. In this image, the fetus 408 is not obstructed by the placenta. FIG. 4B is a maternal image 404 identified by the system 100 as representative of low lying placenta condition. In this image, the fetus 410 is above the placenta which is attached at the edge of the cervix. FIG. 4C is a maternal image 406 identified by the system 100 as representative of previa placenta condition. The fetus 412 is blocked by the placenta in this image. Illustrated images demonstrates efficient working of the proposed scheme for detecting the placenta condition.

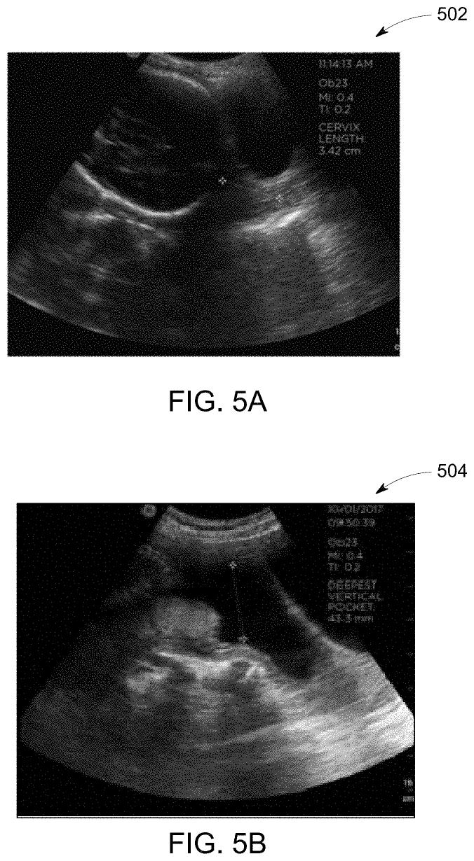

FIGS. 5A, 5B illustrate maternal images representative of classes of maternal images classified by the system 100 of FIG. 1 in accordance with one aspect of the present specification. The detector network used for classifying the maternal images is similar to the structure illustrated in FIG. 3. Suitable network parameters of the classifier are selected and/or determined during training phase. FIG. 5A is a first maternal image 502 classified by the system 100 as a sagittal image. FIG. 5B is a second maternal image 504 classified by the system of 100 as a non-sagittal image. In this example, the classifier network is a learning network designed apriori using a plurality of annotated maternal images. The input layer is configured to receive a two-dimensional maternal image to generate plurality of feature maps. The classifier network is trained using four hundred maternal images having two hundred seventy five sagittal planes and one hundred and twenty five non-sagittal images. The classifier network is configured to generate a binary value representative of a maternal image category. The classifier network is trained using 75% of the maternal images and validated using remaining 25% of the maternal images. The proposed technique could classify the maternal images with 75% accuracy.

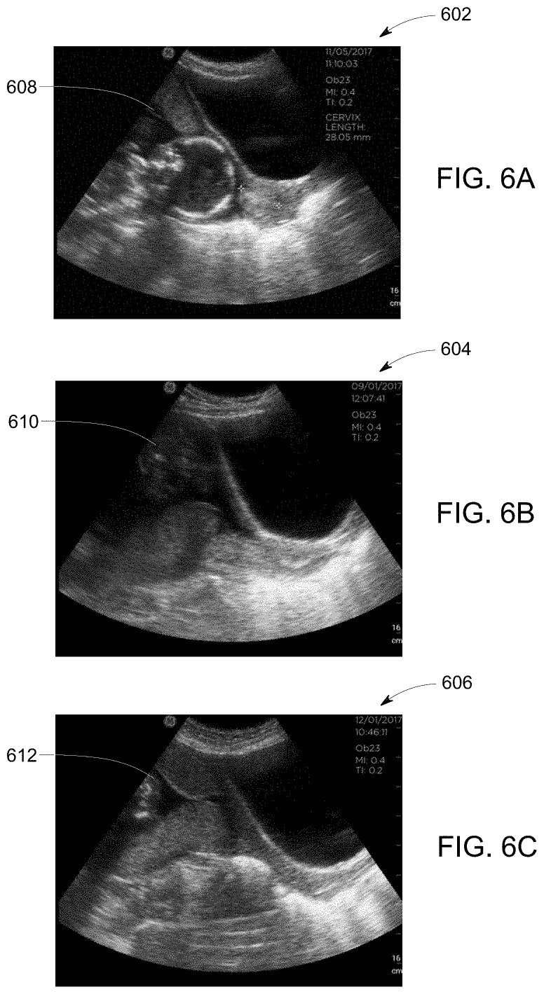

FIGS. 6A-6C illustrate maternal images representative of fetal conditions detected by the system 100 of FIG. 1 in accordance with another aspect of the present specification. The detector network used for determining the placenta condition is similar to the structure illustrated in FIG. 3. Suitable network parameters of the detector network are selected and/or determined during training phase. In this example, the detector network is a learning network designed/generated apriori using a plurality of annotated sagittal images. FIG. 6A is maternal image 602 identified by the system 100 as representing a cephalic condition. The top portion of head of the fetus is represented by numeral 608. FIG. 6B is a maternal image 604 identified by the system 100 as representing a breach condition. The buttock portion of the fetus is indicated by numeral 610. FIG. 6C is a maternal image 606 identified by the system 100 as representing a traverse condition with the fetus indicated by numeral 612. A convolution neural network with multiple convolution stages is used to realize the detector network. The detector network is configured to generate a discrete value representative of a fetal condition. The detector network is trained using two hundred and forty sagittal images annotated with fetal conditions. The sagittal images include images with cephalic condition and one hundred and sixteen images include non-cephalic fetal condition. A detection accuracy of about 69% is achieved after training the network using 75% of the annotated sagittal images and validated with 25% of the rest of sagittal images.

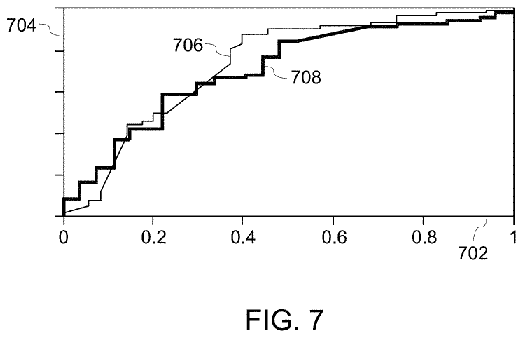

FIG. 7 is a graph 700 illustrating performance of deep learning networks used for realizing classifier network and detector network in accordance with another aspect of the present specification. The graph 700 includes an x-axis 702 representative of a false positive rate on a scale of zero to one. The graph 700 also includes a y-axis 704 representative of a true positive rate on a scale of zero to one. The graph 700 further includes a performance curve 706 corresponding to a classifier network. The performance curve 706 is representative of receiver operating characteristics (ROC) and an area under the curve (AUC) parameter corresponding to the ROC representing quantification of the performance of the classifier network. Each point on the performance curve 706 is determined by training the classifier network using a plurality of maternal images having images both in the sagittal plane and in the near vicinity of the sagittal plane. In one example, one thousand seven hundred and twenty maternal images having nine hundred twenty seven mid sagittal images and seven hundred ninety three images corresponding to planes in the near vicinity of the mid-sagittal plane are used to train the classifier network. About 75% of the images are used for training the classifier network and the remaining 25% of the images are used for validating the trained classifier network. The AUC corresponding to the performance curve 706 is 85%.

The graph 700 further includes a performance curve 708 corresponding to a detector network. The performance curve 708 is representative of receiver operating characteristics (ROC) of the detector network. An area under the curve (AUC) parameter corresponding to the ROC is representative of quantification of the performance of the detector network. Each point on the performance curve 708 is obtained by training the detector network using a plurality of sagittal images having images with various placenta conditions. In one example, nine hundred twenty seven sagittal images having six hundred fifty eight sagittal images corresponding to clear placenta condition and two hundred and sixty nine sagittal images corresponding to placenta complications are used to train the detector network. The sagittal images corresponding to the placenta complications include images corresponding to low-lying, marginal and previa conditions. About 75% of the sagittal images are used for training the detector network and the remaining 25% of the sagittal images are used for validating the trained detector network. The AUC corresponding to the performance curve 708 is 75%.

It is to be understood that not necessarily all such objects or advantages described above may be achieved in accordance with any particular embodiment. Thus, for example, those skilled in the art will recognize that the systems and techniques described herein may be embodied or carried out in a manner that achieves or improves one advantage or group of advantages as taught herein without necessarily achieving other objects or advantages as may be taught or suggested herein.

While the technology has been described in detail in connection with only a limited number of embodiments, it should be readily understood that the specification is not limited to such disclosed embodiments. Rather, the technology can be modified to incorporate any number of variations, alterations, substitutions or equivalent arrangements not heretofore described, but which are commensurate with the spirit and scope of the claims. Additionally, while various embodiments of the technology have been described, it is to be understood that aspects of the specification may include only some of the described embodiments. Accordingly, the specification is not to be seen as limited by the foregoing description, but is only limited by the scope of the appended claims.

1. A system, comprising:
a memory unit comprising a classifier network and a detector network, wherein the classifier network is configured to perform a classification of a scan image among maternal images, and wherein the detector network is configured to determine a placenta condition in the scan image;
a data acquisition unit communicatively coupled to an ultrasound scanner and configured to receive maternal images from a maternal scanning procedure;
an image processing unit communicatively coupled to the memory unit and the data acquisition unit and configured to:
select a sagittal image from the maternal images using the classifier network;
determine a placenta condition based on the selected sagittal image using the detector network; and
provide a recommendation to a medical professional based on the placenta condition.

2. The system of claim 1, further comprising:
a metric network configured to determine a dimensional parameter, stored in the memory unit, wherein the dimensional parameter is representative of at least one of a cervix length and a distance between the placenta and the cervix opening; and
the image processing unit further configured to:
process the sagittal image using the metric network to determine a length value as the dimensional parameter; and
present the length value to the medical professional.

3. The system of 2, wherein the classifier network comprises a first convolution neural learning network, the detector network comprises a second convolution neural learning network and the metric network comprises a third convolution neural learning network.

4. The system of claim 3, wherein the image processing unit is further configured to:
receive a plurality of labelled sagittal images from a database, wherein each of the plurality of labelled sagittal images is annotated with a value of a dimensional parameter;
train the third convolution neural learning network based on a first subset of the plurality of labelled sagittal images;
validate the third convolution neural learning network based on a second subset of the plurality of labelled sagittal images; and
store the validated third convolution neural learning network as the metric network in the database.

5. The system of claim 3, wherein the image processing unit is further configured to:
receive a plurality of labelled maternal images from a database, wherein each of the plurality of labelled maternal images is classified as one of a sagittal image and a non-sagittal image;
train the first convolution neural learning network based on a first subset of the plurality of labelled maternal images;
validate the first convolution neural learning network based on a second subset of the plurality of labelled maternal images; and
store the validated first convolution neural learning network as the classifier network in the database.

6. The system of claim 3, wherein the image processing unit is further configured to:
receive a plurality of labelled sagittal images from a database, wherein each of the plurality of labelled sagittal images is annotated with a fetal condition;
train the second convolution neural learning network based on a first subset of the plurality of labelled sagittal images;
validate the second convolution neural learning network based on a second subset of the plurality of labelled sagittal images; and
store the validated second convolution neural learning network as the detector network in the database.

7. The system of claim 1, wherein the detector network is configured to detect a fetal position corresponding to a breach condition, a cervical competence condition, or both by using the detector network based on the sagittal image.

8. The system of claim 1, wherein the image processing unit is further configured to identify a maternal image corresponding to a plane substantially parallel to a mid-sagittal plane.

9. The system of claim 1, wherein the image processing is configured to:
classify each of the maternal images as a sagittal image or as a non-sagittal image using the classifier network;
determine a metric value representative of an angle formed by a plane represented by each of the maternal images with mid-sagittal plane; and
assist acquisition of a sagittal image based on the metric value.

10. A method, comprising:
receiving maternal images from a maternal scanning procedure;
obtaining a classifier network and a detector network from a memory unit, wherein the classifier network is configured to perform a classification of a scan image among the maternal images, and wherein the detector network is configured to determine a placenta condition in the scan image;
selecting a sagittal image from the maternal images using the classifier network;
determining a placenta condition based on the selected sagittal image using the detector network; and
providing a recommendation to a medical professional based on the placenta condition.

11. The method of claim 10, further comprising:
obtaining, from the memory unit, a metric network configured to determine a dimensional parameter, wherein the dimensional parameter is representative of at least one of a cervix length and a distance between the placenta and the cervix opening;
processing the sagittal image using the metric network to determine a length value as the dimensional parameter; and
presenting the length value to a medical professional.

12. The method of claim 11, wherein the classifier network comprises a first convolution neural learning network, the detector network comprises a second convolution neural learning network and the metric network comprises a third convolution neural learning network.

13. The method of claim 12, wherein obtaining the classifier network comprises:
receiving a plurality of labelled maternal images from a database, wherein each of the plurality of labelled maternal images is classified as one of a sagittal image and a non-sagittal image;
training the first convolution neural learning network based on a first subset of the plurality of labelled maternal images;
validating the first convolution neural learning network based on a second subset of the plurality of labelled maternal images; and
storing the validated first convolution neural learning network as the classifier network in the database.

14. The method of claim 12, wherein obtaining the detector network comprises:
receiving a plurality of labelled sagittal images from a database, wherein each of the plurality of labelled sagittal images is annotated with a fetal condition;
training the second convolution neural learning network based on a first subset of the plurality of labelled sagittal images;
validating the second convolution neural learning network based on a second subset of the plurality of labelled sagittal images; and
storing the validated second convolution neural learning network as the detector network in the database.

15. The method of claim 12, wherein obtaining the metric network comprises:
receiving a plurality of labelled sagittal images from a database, wherein each of the plurality of labelled sagittal images is annotated with a value of a dimensional parameter;
training the third convolution neural learning network based on a first subset of the plurality of labelled sagittal images;
validating the third convolution neural learning network based on a second subset of the plurality of labelled sagittal images; and
storing the validated third convolution neural learning network as the metric network in the database.

16. The method of claim 10, further comprising detecting a fetal position corresponding to a breach condition, a cervical competence condition, or both by using the detector network based on the sagittal image.

17. The method of claim 10, wherein selecting the sagittal image comprises identifying a maternal image corresponding to a plane substantially parallel to a mid-sagittal plane.

18. The method of claim 10, wherein selecting the sagittal image comprises:
classifying each of the maternal images as a sagittal image or as a non-sagittal image using the classifier network;
determining a metric value representative of an angle formed by a plane represented by each of the maternal images with mid-sagittal plane; and
assisting acquisition of a sagittal image based on the metric value.

19. A non-transitory computer readable medium having instructions to enable at least one processor unit to:
receive maternal images from a maternal scanning procedure;
obtain a classifier network and a detector network from a memory unit, wherein the classifier network is configured to perform a binary classification of a scan image among the maternal images, and wherein the detector network is configured to determine a placenta condition in the scan image;
select a sagittal image from the maternal images using the classifier network;
determine a placenta condition based on the selected sagittal image using the detector network; and
provide a recommendation to a medical professional based on the placenta condition.
